(19) United States
(12) Patent Application Publication
(10) Pub. No.: US 2010/0211110 A1
(43) Pub. Date: Aug. 19, 2010

(54) CRANIOFACIAL PLATING SYSTEM AND METHOD

(76) Inventor: Peter D. Costantino, Armonk, NY (US)

Correspondence Address: THOMPSON HINE L.L.P.;Intellectual Property Group, P.O. BOX 8801, DAYTON, OH, 45401-8801, US

(21) Appl. No.: 12/370,785

(22) Filed: Feb. 13, 2009

(51) Int. Cl. A61B 17/80; A61B 17/88

(52) U.S. Cl. 606/286; 606/281

(57) ABSTRACT

A craniofacial plating member comprises a fastening member configured and dimensioned to extend over two portions of bone to be mended together. The fastening member defines a plurality of plate holes, at least a first of the plate holes being positioned to be placed over a first bone hole defined or to be defined in one of the bone portions and a second of the plate holes being positioned to overlie a suitable place for a second bone hole defined or to be defined in the other of said bone portions. They first plate hole overlies the first bone hole, and an attachment member is securely positioned in the first plate hole. In accordance with the preferred embodiment, the attachment member is a pin. That pin further comprises a retention member secured to and integral with the pin. The retention member may also further include a barb.

### TECHNICAL FIELD

The invention provides a specialized plate for mending bone structures particularly useful for application as a craniofacial plate, and a method of using the same.

### STATEMENT REGARDING FEDERALLY SPONSORED RESEARCH OR DEVELOPMENT

(Not applicable)

### BACKGROUND OF THE INVENTION

Metal plates are commonly used by surgeons for mending bone structures where less invasive techniques are likely to be unreliable or where such techniques, for various reasons, may not be available. One application where metal plates are of particular importance is craniofacial surgery. In such applications, additional challenges are presented, in many cases, on account of the proximity of the brain and other sensitive organs.

Generally, metal plates are used to fix the position of a fragment of bone with respect to a secured portion of bone. Such metal plates typically have a number of holes which are positioned at a number of points on the plate calculated to serve as anchor points in a plate deployment configuration designed to secure a portion of unanchored bone to an anchored portion of bone. In use, each anchor point is associated with a screw which, in use, passes through the hole associated with the anchor point and into bone. The plate thus acts as a structural member, rigidly secured to both the anchored and unanchored bone portions. This structural member thus maintains the relative position between the anchored and unanchored bone portions. Multiple anchor points are associated with both the anchored portion of bone and the portion of bone to be secured to the anchored portion of bone. In principle, an additional unsecured bone fragment or fragments may be secured to an other bone fragment after such other bone fragment has been secured.

Multiple anchor points with respect to both the unanchored and anchored portions of bone insure relative stability in the deployment of bone structures. This insures the required mechanical stability needed while the bone heals, and achieves a relatively high degree of mechanical strength, which may be regarded as important to increase the likelihood that the reconstructed bone will not move, or, worse, come apart during the healing process. Typical procedures thus involve use of plates with multiple anchor points for both portions of bone and, moreover, a number of such plates with multiple anchor points positioned to be secured to both portions of bone to be secured to each other.

Typically, craniofacial plating systems employ screws with specialized heads which allow them to be securely held by a specialized mating screwdriver. Once the screws have been mounted on the screwdriver, the surgeon need only grasp the screwdriver in a conventional fashion, and navigate the tip of the screwdriver in such a manner that the tip of the screw may be introduced into and in alignment with a hole in the bone portion into which the screw is to be introduced. More particularly, after the hole in the bone has been drilled, one of the holes in the plate is positioned over the hole in the bone by, for example, a nurse who is holding the plate with a forceps. After that, the surgeon introduces the screw and hand turns the specialized screwdriver to securely seat the screw in the bone.

### SUMMARY OF THE INVENTION

In the case of craniofacial surgery, the surgeon is assisted by a nurse or other professional due to the precision and stability with which the plate must be held, as a rotating screw may tend to apply torque to an insecurely held metal plate. The result may be that

In accordance with the instant invention, a simplified procedure and a higher degree of control is provided for the mechanical parts and tools involved in the repair of fractured and unsecured bone structures. The inventive plating system is particularly useful in craniofacial applications. Moreover, the inventive plating system has the advantage of working well with existing specialized surgical screw drivers, and specialized head surgical screws, while also involving relatively minimal changes in the procedure, thus facilitating adoption of the technique by surgeons and minimizing the learning time and possibility of error, both of which are typically associated with the adoption of new improved techniques by even skilled surgeons.

In addition, the inventive plating system and method increases control to the point where performance of a cranial plating repair procedure by a single unassisted physician becomes a more acceptable option for certain procedures.

In accordance with the invention, a craniofacial plating member comprises a fastening member configured and dimensioned to extend over two portions of bone to be mended together. The fastening member defines a plurality of plate holes, at least a first of the plate holes being positioned to be placed over a first bone hole defined or to be defined in one of the bone portions and a second of the plate holes being positioned to overlie a suitable place for a second bone hole defined or to be defined in the other of said bone portions. The first plate hole overlies the first bone hole, and an attachment member is securely positioned in the first plate hole. In accordance with the preferred embodiment, the attachment member is a pin. That pin further comprises a retention member secured to and integral with the pin. The retention member may also further include a barb.

The inventive craniofacial plating method, comprises placing a plate to extend over first and second portions of bone to be mended together, and placing a first attachment member secured to the plate in a position overlying a first bone hole position on the first bone portion. The first hole is drilled at said first bone hole position on the first bone portion. The first attachment member is inserted into the first bone hole. A plate hole defined in the plate is placed over the second bone portion to be secured to the first bone portion and a hole is drilled in the second bone portion. A second attachment member is placed through the plate hole and into the second bone portion.

In accordance with an alternative embodiment of the inventive craniofacial plating system, the plate may be curved to conform to the curved shape of a bone structure to be repaired, for example the skull.

### BRIEF DESCRIPTION OF THE DRAWINGS

Illustrative embodiments of the inventive craniofacial plate and ways of making and using the invention, as well as the best mode contemplated of carrying out the invention, are described in detail below, by way of example, with reference to the accompanying drawings, in which:

FIG. 1 is a top view of a human skull illustrating a whole to be mended by the improved craniofacial plate;

FIG. 2 is a plan view of the inventive craniofacial plate;

FIG. 3 is a perspective view of a bone fragment to repair the whole illustrated in the skull illustrated in FIG. 1;

FIG. 4 is an isometric view of a specialized surgical screwdriver useful in accordance with the inventive method and plating system; FIG. 5 is a perspective view of the inventive craniofacial plate being moved into position by a specialized surgical screwdriver;

FIG. 6 is a perspective view showing the inventive craniofacial plate being moved into position over a human skull;

FIG. 7 is a perspective you illustrating the placement of the inventive craniofacial plate in a human skull;

FIG. 8 is a perspective view showing the inventive craniofacial plate in position after initial placement in accordance with the method of the present invention;

FIG. 9 is a perspective view of a specialized surgical screwdriver head for engaging a screw to be deployed in accordance with the method of the present invention;

FIG. 10 is a top view of a human skull showing successful attachment of the craniofacial plate of the present invention at two positions;

FIG. 11 is a view similar to FIG. 11 showing a plurality of deployed craniofacial plates;

FIG. 12 is a side view of an alternative sheet-metal member for use in forming a craniofacial plate of the present invention;

FIG. 13 is a top view of the sheet-metal member of FIG. 12;

FIG. 14 is a side view of the alternative craniofacial plate constructed in accordance with the invention;

FIG. 15 is a top view of the craniofacial plate of FIG. 14;

FIG. 16 is a side view of a pin useful in the craniofacial plate of the invention;

FIG. 17 is a side view of the pin of FIG. 16 with the arms flexed;

FIG. 18 illustrates flexure of the arms of the pin of FIG. 16.

FIG. 19 illustrates placement of an inventive craniofacial plate in bone; and

FIG. 20 is a side view of yet another alternative embodiment of the present invention.

### DETAILED DESCRIPTION OF THE DRAWINGS

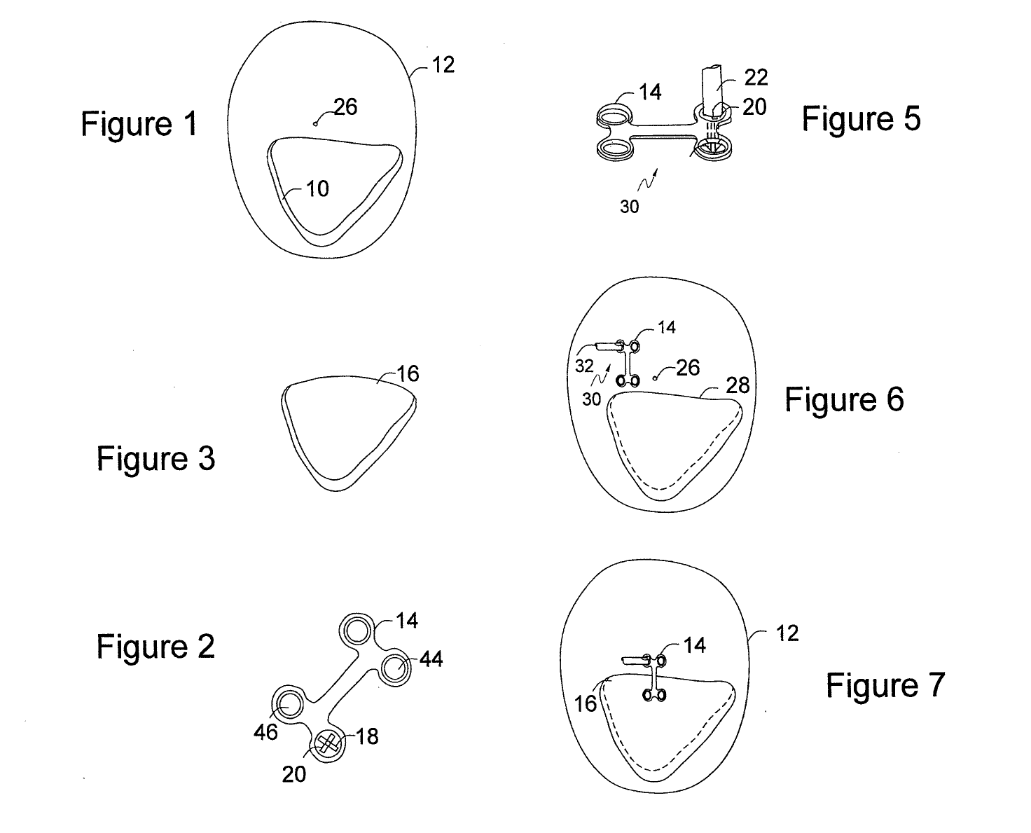

Referring to the figures, and FIG. 1, in particular, a method of closing a hole 10 in a human skull 12 is illustrated. Initially, and optionally, in accordance with the invention the surgeon grasps the inventive plate 14, illustrated in FIG. 2, with the existing specialized screwdriver, meant for specialized bone mending plate screws, and moves it about the area to be repaired to determine a suitable position for plate 14, with position being determined by the strength of the bone, the position of holes on the selected plate 14, the bone shapes involved, and so forth. Optionally, and preferably, such survey is performed after the bone fragment to be secured is positioned in place. In the illustrated example, fragment 16, as illustrated in FIG. 3, is positioned in hole 10 in skull 12, as illustrated, for example, in FIG. 1. In the course of this survey, in particular in unusual circumstances, an initial selection of a particular type or shape of plate may be changed.

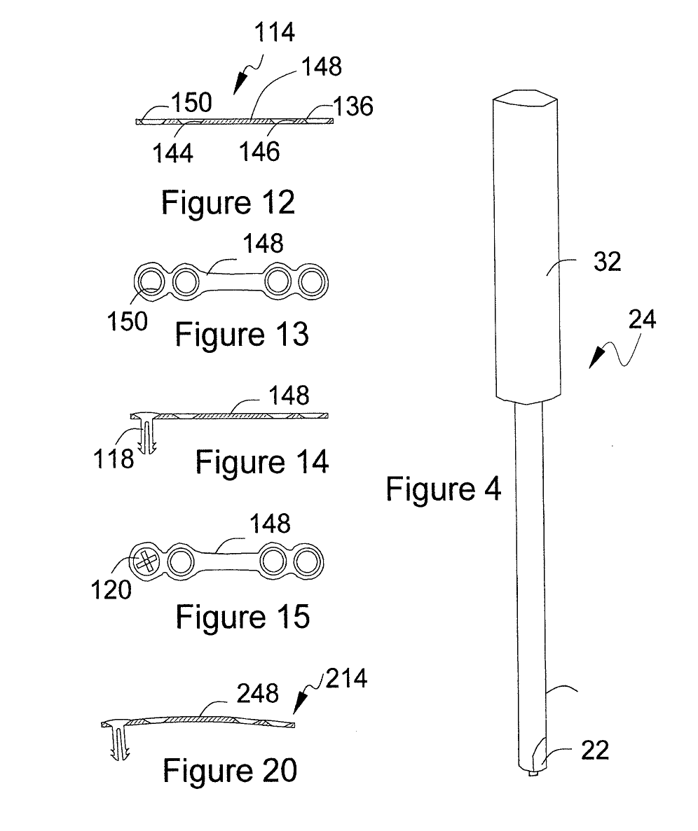

Plate 14 includes a specialized pin 18, which includes engagement structure 20, which is adapted to mate with the head 22 of specialized surgical screwdriver 24, which is illustrated in FIG. 4. In accordance with the invention, the surgeon visually determines the position of plate 14 by moving it to the proposed location and visually verifying the appropriateness of such position. Once the position of the plate has been determined, the position of pin 18 is noted. This may be done by visually identifying the position by a feature of the anatomy, or by any suitable marking mechanism. A hole 26 is then drilled in skull 12 at the position determined for pin 18, as illustrated in FIG. 1.

At this point, fragment 16 is positioned in the hole 10 on skull 12, if this has not already been done. Plate 14 is then grasped by screwdriver 24 through the use of specialized engagement structure 20 on plate 14. The result is to form a substantially unitary workpiece 30 consisting of the shaft 32 of screwdriver 24 and plate 14. More particularly, the head 22 of screwdriver 24 is brought into engagement with specialized engagement structure 20 of plate 14, as illustrated in FIG. 5. Plate 14 is then moved into position, as illustrated in FIG. 6, until plate 14 overlies the interface 28 between skull 12 and fragment 16, as illustrated in FIG. 7, with pin 18 overlying and in alignment with holes 26 in skull 12.

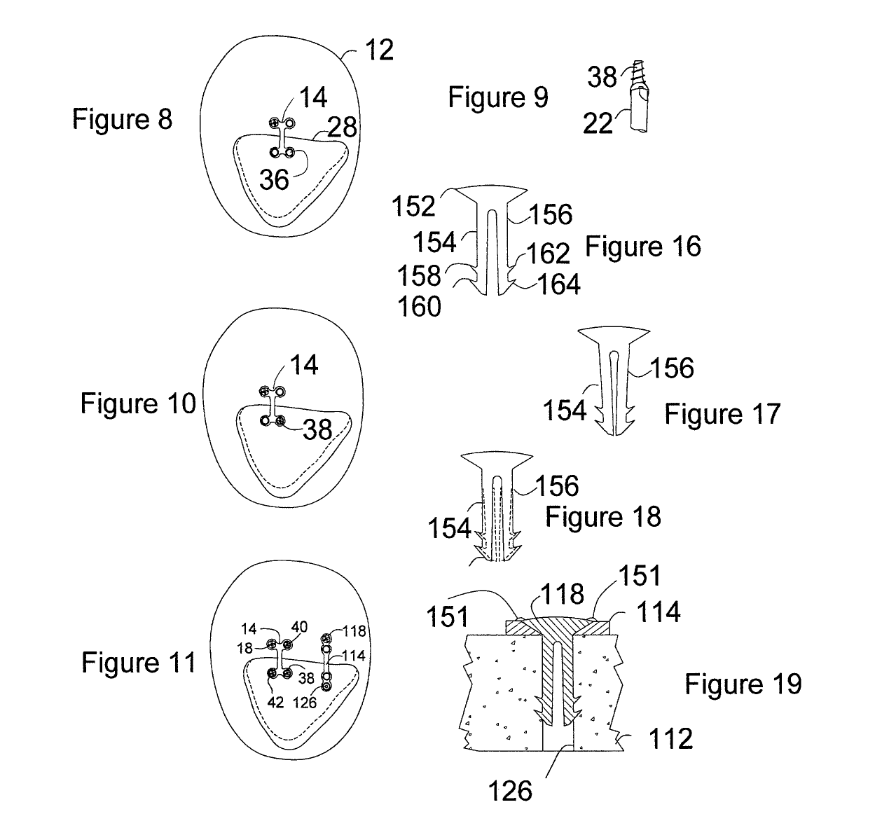

After such alignment has been achieved, the surgeon, grasping handle 32 of surgical screwdriver 24, applies pressure driving pin 18 into hole 26. On account of the specialized structure of pin 18, as will be described in detail below, the result is to securely mount plate 14 in skull 12. Screwdriver 24 is then disengaged from specialized engagement structure 20. Upon such disengagement, plate 14 is mounted in position, as illustrated in FIG. 8.

Following this, a drill is introduced into the hole 36 of plate 14 as illustrated in FIG. 8. The drill is used to drill a hole into the bone in order to receive a surgical screw to further secure plate 14. A conventional surgical screw 38 is then engaged by specialized head 22 of surgical screwdriver 24, as illustrated in FIG. 9. The surgeon then introduces screw 38 into the hole drilled through hole 36 and rotates the handle 32 of screwdriver 24 in a conventional manner to secure screw 38 in fragment 16.

In similar fashion to that of screw 38, screws 40 and 42 are placed into the remaining holes 44 and 46 of plate 14. The result is that plate 14 is securely held in position by screws 38, 40 and 42 acting together with pin 18, as is illustrated in FIG. 11.

As alluded to above, a craniofacial plate constructed in accordance with the president mentioned may take any one of numerous configurations and analogous to the configurations of surgical plates used in the prior art. More particular he, the plate may take the form of an elongated four hole plate of the type illustrated in FIGS. 12-15. More perfectly, as illustrated in FIG. 12, such a plate 114 may be characterized by a plurality of countersunk holes 136, 144, and 146 in a planar sheet metal member 148. Planar sheet metal member 148 may be of a conventional craniofacial plate material, such as titanium, or other suitable material. Planar sheet metal member 148 also defines a hole 150 in which a pin 118 may be mounted.

Pin 118 has specialized engagement structure 120 substantially identical to that in specialized surgical screws which are adapted to be engaged by a screwdriver such as the surgical screwdriver 24 illustrated in FIG. 4. While other fabrication techniques may be use, in accordance with the present invention, it is contemplated that pin 118 would be made of metal such as titanium or any other suitable material, such as plastic. Pin 118, in accordance with the preferred embodiment, is welded into hole 150 at welding points 151 as illustrated in FIG. 19. Thus, using a conventional surgical screwdriver, it is possible to engage specialized engagement structure 120 with the head of a specialized surgical screwdriver such as head 22 of screwdriver 24.

As illustrated in FIG. 16, screw 138 comprises a head 152 and a pair of optionally flexible arms 154 and 156, which support barbs 158, 160, 162 and 164. Flexible arms 154 and 156 are free to flex toward each other, as illustrated in FIG. 17. The relative positions between the flexed and unflexed position is more clearly seen with reference to FIG. 18 where the flexed position is illustrated in dashed lines.

As may be seen most clearly with reference to FIG. 19, the combination of barbs 158-164 and flexible arms 154 and 156 results in secure engagement in hole 126 of the bone forming skull 112.

Referring to FIG. 11, a plate such as that illustrated in FIG. 14 may be deployed on a human skull, by pushing pin 118 into the position as illustrated. Following that, a hole 126 is drilled in fragment 16 allowing placement of additional screws with screwdriver 24.

Referring to FIG. 20, an alternative embodiment of the craniofacial plate is illustrated. Here craniofacial plate 214 is formed from a curved sheet metal member 248. This plate has the advantage of conforming to the curve of the bone. Alternatively, a plate such as that illustrated in FIG. 14 may be bent by hand by the surgeon to conform to the particular task.?

The deployment of craniofacial plate 114, as illustrated in FIG. 11, is similar to the deployment of craniofacial plate 14. As the position is located and a hole 126 for the pin 118 has been drilled, pin 118 is grasped using surgical screwdriver 24, and the arms 154 and 156 are driven into hole 126. The result is that barbs 158-164 securely engage the bone of skull 112, as illustrated in FIG. 19.

While an illustrative embodiment of the invention has been described, it is, of course, understood the modifications will be apparent to those of ordinary skill in the art without departing from the spirit and scope of the invention. For example, a hole 10 such as that illustrated in FIG. 1 would typically be produced in the course of cranial surgery, but the invention may also find application in other surgeries and in reconstructive applications. Such uses are within the scope of the invention which is limited and defined only by the appended claims.

1. A craniofacial plating member, comprising a fastening member configured and dimensioned to extend over two portions of bone to be mended together, said fastening member defining a plurality of plate holes, at least a first of said plate holes being positioned to be placed over a first bone hole defined or to be defined in one of said bone portions and a second of said plate holes being positioned to overlie a suitable place for a second bone hole defined or to be defined in the other of said bone portions while said first plate hole overlies said first bone hole, and an attachment member secured in said first plate hole.

2. A craniofacial plating member as in claim 1, wherein said attachment member is a pin.

3. A craniofacial plating member as in claim 2, wherein said attachment member further comprises a retention member secured to and integral with said pin.

4. A craniofacial plating member as in claim 3, wherein said retention member comprises a barb.

5. A craniofacial plating member, comprising a plate configured and dimensioned to extend over two portions of bone to be mended together, said plate defining at least one plate hole, and a first attachment member secured to said plate, said first attachment member being positioned to be placed over a first bone hole defined or to be defined in one of said bone portions and said plate hole being positioned to overlie a suitable place for a second bone hole defined or to be defined in the other of said bone portions while said first attachment member is positioned in said first bone hole, and a second attachment member configured to be secured in said first plate hole.

6. A craniofacial plating member as in claim 5, wherein said first attachment member is a pin.

7. A craniofacial plating member as in claim 6, wherein said first attachment member further comprises a retention member secured to and integral with said pin.

8. A craniofacial plating member as in claim 7, wherein said retention member comprises a barb.

9. A craniofacial plating member as in claim 5, wherein said plate defines a plurality of additional holes configured to receive additional attachment members.

10. A craniofacial plating member, comprising a plate defining at least one plate hole, and a first attachment member secured to said plate, and a second attachment member configured to be secured in said first plate hole.

11. A craniofacial plating member as in claim 10, wherein said first attachment member is a pin.

12. A craniofacial plating member as in claim 11, wherein said first attachment member further comprises a retention member secured to and integral with said pin.

13. A craniofacial plating member as in claim 12, wherein said retention member comprises a barb.

14. A craniofacial plating member as in claim 12, wherein said first attachment member is rotatably mounted on said plate.

15. A craniofacial plating method, comprising:
placing a plate to extend over first and second portions of bone to be mended together,
placing a first attachment member secured to said plate in a position overlying a first bone hole position on said first bone portion, drilling a first hole at said first bone hole position on said first bone portion,
inserting said first attachment member into said first bone hole, positioning a plate hole defined in said plate over said second bone portion to be secured to said first bone portion, drilling a hole in said second bone portion, and
placing a second attachment member through said plate hole and into said second bone portion.

16. A craniofacial plating method, as in claim 15 wherein the method is performed in the following sequence:
said drilling said first hole at said first bone hole position on said first bone portion,
is followed by said placing a first attachment member secured to said plate in a position overlying a first bone hole position on said first bone portion,
which is followed by said inserting said first attachment member into said first bone hole,
which is followed by said positioning said plate hole defined in said plate over said second bone portion to be secured to said first bone portion,
which is followed by said drilling said hole in said second bone portion, and
which is followed by said placing said second attachment member through said plate hole and into said second bone portion.

17. A craniofacial plating method, as in claim 16, wherein said positioning of said plate hole defined in said plate over said second bone portion to be secured to said first bone portion is performed by rotation of said plate about said first attachment member.

18. A craniofacial plating method, as in claim 1, wherein said pin is pushed into said first bone hole, and said first bone hole is made in the more secure of said bone portions.

19. A craniofacial plating member as in claim 1, wherein a second of said plate holes is positioned to overlie a suitable place for a second bone hole defined or to be defined in the other of said bone portions while said first plate hole overlies said first bone hole, and an attachment member secured in said first plate hole.
